(19) United States
(12) Patent Application Publication
(10) Pub. No.: US 2026/0140279 A1
(43) Pub. Date: May 21, 2026

(54) METHOD FOR TRACKING FOUR-DIMENSIONAL SOIL MOISTURE DROUGHT EVENT AND IDENTIFYING WARMING SIGNAL

(71) Applicants: CHINA UNIVERSITY OF GEOSCIENCES (WUHAN), Wuhan (CN); SHENZHEN RESEARCH INSTITUTE OF CHINA UNIVERSITY OF GEOSCIENCES, Shenzhen (CN)

(72) Inventors: Xihui GU, Wuhan (CN); Yansong GUAN, Wuhan (CN); Lunche WANG, Wuhan (CN); Shengzhi HUANG, Wuhan (CN); Xiang ZHANG, Wuhan (CN); Qian CAO, Wuhan (CN); Zengliang LUO, Wuhan (CN); Gang WANG, Wuhan (CN)

(73) Assignees: CHINA UNIVERSITY OF GEOSCIENCES (WUHAN), Wuhan (CN); SHENZHEN RESEARCH INSTITUTE OF CHINA UNIVERSITY OF GEOSCIENCES, Shenzhen (CN)

(21) Appl. No.: 19/452,241

(22) Filed: Jan. 17, 2026

(51) Int. Cl. G01W 1/10; G01N 33/24; G01W 1/02; G01W 1/14

(52) U.S. Cl. CPC G01W 1/10; G01N 33/246; G01W 1/02; G01W 1/14; G01W 2201/00

(57) ABSTRACT

A method for tracking a four-dimensional soil moisture drought event and identifying a warming signal, includes following steps: data acquisition; identification of four-dimensional soil moisture drought events; characteristic quantification of four-dimensional soil moisture drought events; anthropogenic warming signal detection based on temporal evolution of four-dimensional soil moisture drought characteristics in a historical period; estimation of future evolution of four-dimensional soil moisture drought; and attribution of four-dimensional soil moisture drought events to driving factors. The future spatial-temporal evolution characteristics of four-dimensional soil moisture drought are explored, and the driving factors and physical mechanisms of four-dimensional soil moisture drought are analyzed. A strong scientific basis is provided for tackling global climate change, safeguarding water resource security, and formulating disaster prevention and mitigation strategies.

### TECHNICAL FIELD

The present disclosure relates to the field of carbon neutrality, and in particular, to a method for tracking a four-dimensional soil moisture drought event and identifying a warming signal.

### BACKGROUND

Soil moisture drought, characterized by soil moisture deficit, is one of the most severe natural disasters, exerting adverse impacts on water resources, ecosystems, plant growth, and crop yields. Affected by climatic factors, vegetation, topography, soil properties, and land utilization, the variation of soil moisture differs across soil depths on a regional scale. Water stored in deep soil layers can be directly utilized for vegetation growth. Therefore, moisture deficit in deep soil causes greater damage to plants and agricultural production than that in surface soil. Considering that vegetation exhibits high spatial heterogeneity and significant variations in root depth, understanding the changes in the vertical structure of global soil moisture drought is crucial for more accurate monitoring and assessment of its adverse impacts on ecosystems. Against the backdrop of global warming, emissions such as greenhouse gases are increasingly aggravating soil moisture drought. Currently, there is no detailed technology exploring the variation of soil moisture drought from the perspective of continuous evolution in four dimensions (longitude, latitude, depth, and time), and it remains unclear whether such a variation is affected by climate change induced by anthropogenic warming.

### SUMMARY OF PRESENT INVENTION

An objective of the present disclosure is to provide a method for tracking a four-dimensional soil moisture drought event and identifying a warming signal to solve the technical problem of no detailed studies on the spatial-temporal evolution of global soil moisture drought vertical structures.

The present disclosure provides a method for tracking a four-dimensional soil moisture drought event and identifying a warming signal, including following steps:
step S1, data acquisition: acquiring global climate data;
step S2, identification of four-dimensional soil moisture drought events: calculating, in combination with the data obtained in the step S1, a 10th percentile of a soil moisture value during a growing season of a climatological period for each voxel (each grid-cell×depth-layer unit) as a drought threshold, identifying voxels for which soil moisture values are lower than the drought threshold as drought voxels, and identifying continuous four-dimensional soil moisture drought events by a Lagrangian four-dimensional tracking method;
step S3, characteristic quantification of four-dimensional soil moisture drought events: calculating characteristics of soil moisture drought events in combination with the four-dimensional soil moisture drought events obtained in the step S2, and classifying the soil moisture drought events into surface drought and deep drought;
step S4: anthropogenic warming signal detection based on temporal evolution of four-dimensional soil moisture drought characteristics in a historical period: performing anthropogenic warming signal detection on drought characteristic indicators calculated based on reanalysis data and a historical climatic experiment of a climate model by using a correlation coefficient method and an optimal fingerprint method in combination with duration and intensity characteristics of two types of drought events obtained in the step S3;
step S5: estimation of future evolution of four-dimensional soil moisture drought: obtaining spatial-temporal evolution characteristics of drought events in a future period through the historical climatic experiment of the climate model and a future socio-economic development pathway experiment in combination with the duration and intensity characteristics of the two types of drought events obtained in the step S3; and obtaining spatial-temporal evolution characteristics of drought events under different vegetation types in the future period in combination with global land cover data obtained in the step S1; and
step S6: attribution of four-dimensional soil moisture drought events to driving factors: attributing the two types of drought events to three early-stage driving factors according to data of near-surface air temperature, precipitation, and leaf area index obtained in the step S1 in combination with the duration and intensity characteristics of the two types of drought events obtained in the step S3, and quantifying contributions of different driving factors to the drought events.

A storage medium stores instructions and data for implementing a method for tracking a four-dimensional soil moisture drought event and identifying a warming signal.

A device for tracking a four-dimensional soil moisture drought event and identifying a warming signal includes a processor and a storage medium, where the processor is configured to load and execute instructions and data in the storage medium to implement a method for tracking a four-dimensional soil moisture drought event and identifying a warming signal.

The present disclosure has the following beneficial effects:

(1) The present disclosure provides a method for identifying four-dimensional soil moisture drought, which helps to deepen the understanding of this new type of soil moisture drought for the academic community. In the present disclosure, the four-dimensional soil moisture drought is classified into two different types; the spatial-temporal characteristic indicators of the four-dimensional soil moisture drought are quantified; and the changes and impacts of the four-dimensional soil moisture drought in the four-dimensional spatial-temporal domain (including longitude, latitude, depth, and time) are clarified. In the present disclosure, the differences in spatial-temporal evolution of two types of four-dimensional soil moisture drought under different land cover types in future periods are quantified, providing a scientific basis for in-depth understanding of the dynamic evolution process of drought events.

(2) In the present disclosure, quantitative anthropogenic warming signal detection is performed on the spatial-temporal evolution process of the four-dimensional soil moisture drought characteristics, the impacts of anthropogenic warming on the changes of the two types of four-dimensional drought events are identified, and the changes of four-dimensional drought events in the future are estimated. It provides theoretical support for decision-makers to make decisions for adapting to and mitigating the impact of climate change on soil moisture drought.

(3) In the present disclosure, the contributions of the driving factors of the four-dimensional soil moisture drought events are analyzed; the contributions of different driving factors to four-dimensional soil moisture drought in historical and future periods are clarified; the dominant driving factors of the two types of four-dimensional soil moisture drought are quantified; theoretical support is provided for the attribution of four-dimensional soil moisture drought.

### BRIEF DESCRIPTION OF THE DRAWINGS

FIG. 1 is a flowchart of a method of the present disclosure;

FIG. 2 is diagram showing examples of surface drought and deep drought defined with a Lagrangian four-dimensional tracking framework;

FIG. 3 is a diagram showing spatial distributions of centroid of global surface drought and deep drought in a historical period;

FIG. 4 is a diagram showing duration and intensity changes of global surface drought and deep drought in a historical period;

FIG. 5 is a diagram showing trends in durations and intensities vs probability densities for global surface drought and deep drought in a historical period;

FIG. 6 is a diagram showing spatial-temporal evolution of durations of global surface drought and deep drought events in a future period;

FIG. 7 is a diagram showing spatial-temporal evolution of intensities of global surface drought and deep drought events in a future period;

FIG. 8 is a diagram showing variations in durations and intensities of global deep drought and surface drought under different land coverages;

FIG. 9 is a diagram showing contributions of different driving factors to durations of surface drought and deep drought events;

FIG. 10 is a diagram showing contributions of different driving factors to intensities of surface drought and deep drought events;

FIG. 11 is a diagram showing a time series of three driving factors in a growing season in different scenarios; and

FIG. 12 is a schematic diagram of working of a hardware device of the present disclosure.

### DETAILED DESCRIPTION OF THE EMBODIMENTS

In order to make the objective, technical solution, and advantages of the present disclosure clearer, the embodiments of the present disclosure will be further described in detail in conjunction with the accompanying drawings.

Before formally explaining the present disclosure, the solutions of the present disclosure are generally set forth to facilitate understanding.

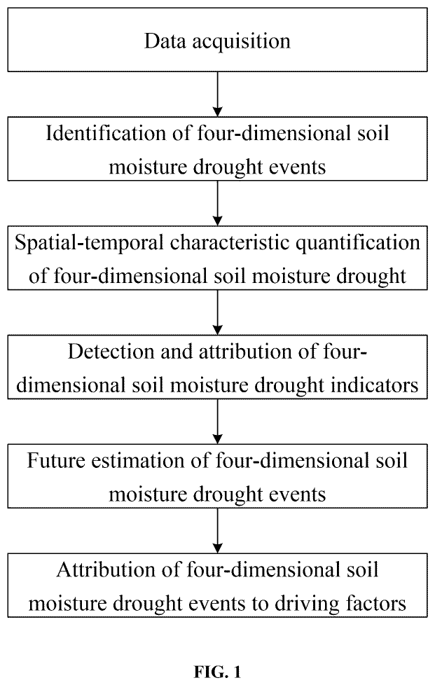

FIG. 1 is a flowchart of a method of the present disclosure.

The present disclosure provides a method for tracking a four-dimensional soil moisture drought event and identifying a warming signal, including following steps.

In step S1, data acquisition is performed: global climate data is acquired.

Specifically, in the present disclosure, the following data is acquired: soil moisture content, near-surface air temperature, and precipitation data from three reanalysis datasets including a fifth generation land surface reanalysis dataset of European Centre for Medium-Range Weather Forecasts (ERA5-Land), global atmosphere and land surface reanalysis product data of China Meteorological Administration (CRA-Land), and Global Land Data Assimilation System Noah model (GLDAS-Noah); leaf area index data of Global Long-Term Leaf Area Index (GLOBMAP) data; precipitation data of Global Soil Moisture Project Phase 3 (GSWP-3); soil moisture content, near-surface air temperature, precipitation, and leaf area index data of Coupled Model Intercomparison Project Phase 6 (CMIP6); and acquired global land cover data of Moderate Resolution Imaging Spectroradiometer (MODIS) Land Cover Climate Modeling Grid (MCD12C1).

In step S2, identification of four-dimensional soil moisture drought events is performed: a 10th percentile of a soil moisture value during a growing season of a climatological period for each voxel (each grid-cell×depth-layer unit) is calculated as a drought threshold; voxels for which soil moisture values are lower than the drought threshold are identified as drought voxels; and continuous four-dimensional soil moisture drought events are identified by a Lagrangian four-dimensional tracking method.

It needs to be noted that in the step S2, soil moisture data is preprocessed: grid cells of Antarctica, Greenland, and desert regions with an annual precipitation of less than 100 mm are removed according to GSWP-3 precipitation data; a soil moisture value in a growing season (May to September in the Northern Hemisphere and November to March of the following year in the Southern Hemisphere) is used; units of the soil moisture value are converted from mass units (kg·m⁻²) to volume units (m³·m³); multi-layer soil moisture values are interpolated to a common vertical profile (ranging from 0 cm to 7 cm, from 7 cm to 28 cm, from 28 to 100 cm, and from 100 cm to 289 cm); datasets on different time scales are converted to datasets on a day scale; the datasets are interpolated with a resolution 1°×1° by second-order conservative remapping; and abnormal sequence fluctuations are eliminated by 11-day smoothing averaging.

Simulation of spatial-temporal evolution of soil moisture drought in four dimensions by using the Lagrangian method is specifically as follows: for each voxel (each grid-cell×depth-layer unit), when the soil moisture value is lower than the 10th percentile of the soil moisture value in the growing season in a climatological period (1981-2010), the voxel is defined as the drought voxel; adjacent drought voxels in the single layer are combined into consecutive drought patches in a two-dimensional space; for each time step, all adjacent two-dimensional drought patches in 26 adjacent areas (nine neighbors above, eight in the same layer excluding the center, and nine below) are joined into an independent three-dimensional consecutive drought patch; and if an overlapping ratio of any spatially consecutive three-dimensional drought patches is greater than 50% in consecutive time steps, the spatially consecutive three-dimensional drought patches are combined into a single four-dimensional (longitude-latitude-depth-time) drought event. The four-dimensional drought event lasts at least for 3 days, and a projection area is greater than 100000 square kilometers.

In step S3, characteristic quantification of four-dimensional soil moisture drought events is performed: characteristics of soil moisture drought events are calculated in combination with the four-dimensional soil moisture drought events obtained in the step S2, and the soil moisture drought events are classified into surface drought and deep drought.

It needs to be noted that in the step S3, spatial and temporal characteristics of the four-dimensional soil moisture drought event are a duration and an intensity, where the duration refers to a duration of the four-dimensional soil moisture drought event; and the intensity is a weighted average of an area and a voxel with soil moisture deficit (threshold minus soil moisture value) among all voxels in the four-dimensional soil moisture drought event, and is expressed as follows:

$$Instensity = \frac{{d}_{l}}{{\sum }_{l = 1}^{k} {d}_{l}} ( {\sum }_{l = 1}^{k} ( \frac{{\sum }_{t = {t}_{0}}^{D} {\sum }_{i = 1}^{{cluster}_{l , t}} ( {thres}_{i , l} - {sm}_{i , l , t} ) \cdot {s}_{i}}{D \cdot {\sum }_{i = 1}^{{cluster}_{l , t}} {s}_{i}} ) )$$

where i represents a voxel; t represents a time; D represents a duration; l represents a soil layer; d represents the thickness of a soil layer; s represents an area of the grid cell; thres represents the drought threshold; sm represents the soil moisture value; and cluster represents a drought event.

These four-dimensional drought events are classified into three drought types, namely surface drought of a heavy-top inverted-iceberg-shaped structure, deep drought of a heavy-bottom iceberg-shaped structure, and a cylindrical drought structure when both surface drought and deep drought do not happen, where the surface drought is defined as that a duration of a surface soil moisture deficit area being larger than a deep soil moisture deficit area is longer than 60% of a total duration; the deep drought is defined as that a duration of the deep soil moisture deficit area being larger than the surface soil moisture deficit area is longer than 60% of the total duration. In the classification process, uppermost two layers (0-7 cm and 7-28 cm) are selected as surface soil, and a third layer (28-100 cm) is selected as deep soil.

In step S4, anthropogenic warming signal detection based on temporal evolution of four-dimensional soil moisture drought characteristics in a historical period: performing anthropogenic warming signal detection on drought characteristic indicators calculated based on reanalysis data and historical climatic experiments of climate models by using a correlation coefficient method and an optimal fingerprint method in combination with duration and intensity characteristics of two types of drought events obtained in the step S3;

It needs to be noted that in the step S4, anthropogenic warming signal detection is performed on the four-dimensional soil moisture drought events based on the correlation coefficient method, and a Spearman correlation coefficient between changes of global surface drought and deep drought characteristic sequences based on reanalysis (an average of three reanalysis datasets) and CMIP6 historical experiment simulation (a multi-model average); a CMIP6 pre-industrial experiment simulation is divided into a plurality of data chunks for same years (40 years) as a research period, random sampling is performed for 2000 times to generate a large number of samples, and the Spearman correlation coefficient between the plurality of data chunks and a multi-model average of historical experiment simulation; and whether a correlation coefficient between the reanalysis data and a drought event characteristic sequence of the historical experiment simulation exceeds a 95th or 99th percentile of a correlation between the historical experiment simulation and 2000 bootstrapping 40-year pre-industrial simulation data chunks. If the correlation between reanalysis and the historical experiment simulation is greater than the 95th or 99th percentile of the correlation between the historical experiment simulation and 2000 bootstrapping 40-year pre-industrial simulation data chunks, it indicates that the changes in the surface and deep drought characteristics exhibit external forcing signals, which may be attributed to anthropogenic warming.

Anthropogenic warming signal detection is performed on the four-dimensional soil moisture drought events based on the optimal fingerprint method, with specific calculation as follows:

$$Y = ( X - α ) β + ε$$

where Y represents a global average characteristic change of surface drought and deep drought based on reanalysis; X represents a global average characteristic change of surface drought and deep drought based on model simulation; α represents sampling uncertainty; β represents a scaling factor; ε represents an internal climatic variability estimated through the pre-industrial experiment simulation. To reduce the noise caused by interannual variations, the characteristic sequences of surface drought and deep drought are averaged at intervals of 3 consecutive years; if both the scaling factor and its 90% confidence interval are greater than zero, the external forcing signal is considered detectable at the 5% significance level. That is, the anthropogenic warming signal can be detected.

In step S5, estimation of future evolution of four-dimensional soil moisture drought is performed: spatial-temporal evolution characteristics of drought events in a future period are obtained through the historical climatic experiment of the climate model and a future socio-economic development pathway experiment in combination with the duration and intensity characteristics of the two types of drought events obtained in the step S3; and spatial-temporal evolution characteristics of drought events under different vegetation types in the future period are obtained in combination with the global land cover data obtained in the step S1.

It needs to be noted that in the step S5, the spatial-temporal evolution of global surface drought and deep drought characteristics in the future period is calculated by using a CMIP6 historical experiment simulation and representative concentration pathway and a shared socio-economic pathway (SSP) experiment simulations in a future scenarios including SSP245 and SSP585 scenarios.

Changes of surface drought and deep drought characteristics of different vegetation types are estimated in combination with a land cover dataset; deep-rooted vegetation cover categories (i.e., forest, shrub, and savannah) are selected, and a weighted area average of deep drought and surface drought characteristic (duration and intensity) differences of different vegetation regions is calculated.

In step S6, attribution of four-dimensional soil moisture drought events to driving factors is performed: the two types of drought events are attributed to three early-stage driving factors according to data of near-surface air temperature, precipitation, and leaf area index obtained in the step S1 in combination with the duration and intensity characteristics of the two types of drought events obtained in the step S3, and contributions of different driving factors to the drought events are quantified.

It needs to be noted that in step S61, surface drought and deep drought events are attributed to different driving factors, such as extreme high temperature (TEM), precipitation deficit (PRE), and vegetation greening (high leaf area index: LAI), an average of three variables in 30 days before a drought event is calculated, and a severity index (SI) of the drought event is calculated, with specific calculation as follows:

$${SI}_{v} = ( {v}_{i , j} - {μ}_{j} ) / {σ}_{j}$$

where i represents a year; j represents a day; v represents a driving factor; and μ and σ represent a climatological normal and a standard deviation of a climatological (historical period: 1981-2010, and future period: 2061-2100) variable, respectively.

In step S62, the extreme high temperature is defined as SI_{TEM}>0.8, the precipitation deficit is defined as SI_{PRE}<−0.8, and the vegetation greening is defined as SI_{LAI}>0.8; if a drought event and an extreme event occurring in 30 days before the drought event appear at a same grid cell, the drought event at the grid cell is attributed to the driving factor of this type; and normalized contributions of different driving factors to durations and intensities of the two types of drought events are calculated.

As an implementation, the present disclosure is further described with the worldwide range and 1981-2020 as an example. The implementation is intended to illustrate the present disclosure, rather than limit an application scope of the present disclosure. The case is also applied to other regions and other time periods.

The flowchart of the method for tracking a four-dimensional soil moisture drought event and identifying a warming signal of the present disclosure is as shown in FIG. 1, and specific steps are as follows.

### (1) Acquisition of Test Data

In this implementation, the hourly soil moisture contents (in units of m³·m⁻³) with the spatial resolution 0.1°×0.1° in 1981-2020 in ERA5-Land, the depths of 0-7 cm, 7-28 cm, 28-100 cm, and 100-289 cm, and the data of near-surface air temperature and precipitation are acquired.

3-Hour soil moisture contents (in units of m³·m⁻³) with the spatial resolution 34 km×34 km in CRA-Land, the depths of 0-10 cm, 10-40 cm, 40-100 cm, and 100-200 cm, and the data of near-surface air temperature and precipitation are acquired.

3-Hour soil moisture contents (in units of kg m⁻²) with the spatial resolution 34 km×34 km in 1981-2014 from GLDAS-Noah edition 2.0 and in 2000-2020 from edition 2.1, the depths of 0-10 cm, 10-40 cm, 40-100 cm, and 100-200 cm, and the data of near-surface air temperature and precipitation are acquired.

The 8 km×8 km leaf area index data is acquired from GLOBMAP. The temporal resolution during 1981-2000 is half a month, and the temporal resolution during 2001-2020 is 8 days.

The precipitation data of GSWP-3 is acquired; and the global land cover data with the spatial resolution 0.05°×0.05° in 2001-2022 of MCD12C1 is acquired.

Furthermore, the experiment simulation data of CMIP6 is acquired. The experiment scenarios include a historical climate experiment, a future socio-economic development pathway experiment, and a pre-industrial experiment. The variables include soil moisture content, near-surface air temperature, precipitation, and leaf area index. The time series are 1981-2020 and 2021-2100, as shown in Table 1 below.

TABLE 1
Basic Information Output by Selected CMIP6 Model

| | | | Pre-Industrial | | |
|---|---|---|---|---|---|
| | | | Experiment | Number | |
| | Set | | Simulation | of | |
| Model Name | Member | Variable | (Year) | Layers | Soil Depth (cm) |
| ACCESS-ESM1-5 | r1i1p1f1 | x₁, x₂, | 1000 | 6 | 1.1, 5.1, 15.7, 43.85, 118.55, |
| | | x₃, x₄ | | | 287.2 |
| CESM2 | r11i1p1f1 | x₁, x₂, | / | 20 | 1, 4, 9, 16, 26, 40, 58, 80, 106, |
| | | x₃, x₄ | | | 136, 170, 208, 250, 299, 358, |
| | | | | | 427, 506, 595, 694, 803 |
| CMCC-CM2-SR5 | r1i1p1f1 | x₁, x₂, | 500 | 15 | 0.71, 2.792, 6.226, 11.887, |
| | | x₃, x₄ | | | 21.219, 36.607, 61.976, 103.803, |
| | | | | | 172.764, 286.461, 473.916, |
| | | | | | 782.977, 1292.532, 2132.647, |
| | | | | | 3517.762 |
| CMCC-ESM2 | r1i1p1f1 | x₁, x₂, | 500 | 15 | 0.71, 2.792, 6.226, 11.887, |
| | | x₃, x₄ | | | 21.219, 36.607, 61.976, 103.803, |
| | | | | | 172.764, 286.461, 473.916, |
| | | | | | 782.977, 1292.532, 2132.647, |
| | | | | | 3517.762 |
| CNRM-CM6-1 | r1i1p1f2 | x₁, x₂, | / | 14 | 0.5, 2.5, 7, 15, 30, 50, 70, 90, |
| | | x₃, x₄ | | | 125, 175, 250, 400, 650, 1000 |
| CNRM-ESM2-1 | r1i1p1f2 | x₁, x₂, | / | 14 | 0.5, 2.5, 7, 15, 30, 50, 70, 90, |
| | | x₃, x₄ | | | 125, 175, 250, 400, 650, 1000 |
| EC-Earth3-CC | r1i1p1f1 | x₁, x₂, | 505 | 4 | 3.5, 17.5, 64, 194.5 |
| | | x₃, x₄ | | | |
| HadGEM3-GC31-LL | r1i1p1f3 | x₁ | / | 4 | 5, 22.5, 67.5, 200 |
| MPI-ESM1-2-HR | r1i1p1f1 | x₁, x₂, | 500 | 5 | 3, 19, 78, 268, 698 |
| | | x₃, x₄ | | | |
| MPI-ESM1-2-LR | r1i1p1f1 | x₁, x₂, | 1000 | 5 | 3, 19, 78, 268, 698 |
| | | x₃, x₄ | | | |
| NorESM2-LM | r1i1p1f1 | x₁, x₂, | 501 | 20 | 1, 4, 9, 16, 26, 40, 58, 80, 106, |
| | | x₃, x₄ | | | 136, 170, 208, 250, 299, 358, |
| | | | | | 427, 506, 595, 694, 803 |
| NorESM2-MM | r1i1p1f1 | x₁, x₂, | 500 | 20 | 1, 4, 9, 16, 26, 40, 58, 80, 106, |
| | | x₃, x₄ | | | 136, 170, 208, 250, 299, 358, |
| | | | | | 427, 506, 595, 694, 803 |
| UKESM1-0-LL | r1i1p1f2 | x₁ | / | 4 | 5, 22.5, 67.5, 200 |

Notes:
x₁, x₂, x₃, and x₄ respectively represent the soil moisture content, near-surface air temperature, precipitation, and leaf area index.

### (2) Identification of Four-Dimensional Soil Moisture Drought Events

In this implementation, the globe is taken as the range. Based on the preprocessing of 3-layer soil moisture values from 3 global reanalysis datasets in 1981-2020, the threshold of the 10th percentile of the soil moisture value during the growing season of the climatological period (1981-2010) is calculated for each voxel (each grid-cell×depth-layer unit). Voxels with soil moisture values lower than the threshold are identified as drought voxels. The growing season is defined as May to September in the Northern Hemisphere and November to March of the following year in the Southern Hemisphere. The four-dimensional continuous drought events are obtained by the Lagrangian method.

Firstly, soil moisture data is preprocessed: grid cells of Antarctica, Greenland, and desert regions with an annual precipitation of less than 100 mm are removed according to GSWP-3 precipitation data; a soil moisture value in a growing season (May to September for the Northern Hemisphere and November to March for the Southern Hemisphere) is used; units of the soil moisture value are converted from mass units (kg m⁻²) to volume units (m³·m³); multi-layer soil moisture values are interpolated to a common vertical profile (ranging from 0 cm to 7 cm, from 7 cm to 28 cm, from 28 to 100 cm, and from 100 cm to 289 cm); datasets on different time scales are converted to datasets on a day scale; the datasets are interpolated with a resolution 1°×1° by second-order conservative remapping; and abnormal sequence fluctuations are eliminated by 11-day smoothing averaging.

Simulation of spatial-temporal evolution of soil moisture drought in four dimensions by using the Lagrangian method is specifically as follows: for each voxel, when the soil moisture value is lower than the 10th percentile of the soil moisture value in the growing season in a climatological period (1981-2010), the voxel is defined as the drought voxel; adjacent drought voxels in the single layer are combined into consecutive drought patches in a two-dimensional space; for each time step, all adjacent two-dimensional drought patches in 26 adjacent areas (nine neighbors above, eight in the same layer excluding the center, and nine below) are joined into an independent three-dimensional consecutive drought patch; and if an overlapping ratio of any spatially consecutive three-dimensional drought patches is greater than 50% in consecutive time steps, the spatially consecutive three-dimensional drought patches are combined into a single four-dimensional (longitude-latitude-depth-time) drought event. In this implementation, the four-dimensional drought event lasts at least for 3 days, and the projection area is greater than 100000 square kilometers.

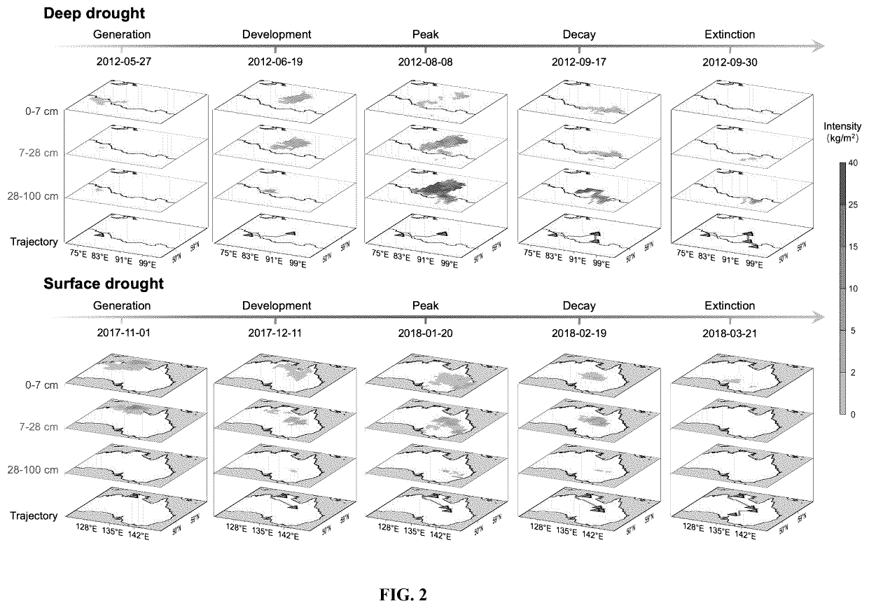

In this implementation, the schematic diagram for determining deep drought and surface drought with the Lagrangian four-dimensional framework is shown in FIG. 2. During the local growing season of 2012, a deep drought event occurred in Central Asia. In 62% of the duration, the drought area of deep soil was larger than that of surface soils. During the local growing season of 2017, the surface drought event in Australia showed that in 86% of the duration, the drought area of surface soils was larger than that of deep soil.

### (3) Quantification of Spatial-Temporal Characteristics of Four-Dimensional Soil Moisture Drought

In this implementation, the characteristics of four-dimensional soil moisture drought are quantified using three reanalysis datasets and the climatic model simulations of CMIP6. The characteristics of the four-dimensional soil moisture drought event are a duration and an intensity, where the duration refers to a duration of the four-dimensional soil moisture drought event; and the intensity is a weighted average of an area and a depth of a grid cell with soil moisture deficit (threshold minus soil moisture value) among all voxels in the four-dimensional soil moisture drought event, and is expressed as follows:

$$Instensity = \frac{{d}_{l}}{{\sum }_{l = 1}^{k} {d}_{l}} ( {\sum }_{l = 1}^{k} ( \frac{{\sum }_{t = {t}_{0}}^{D} {\sum }_{i = 1}^{{cluster}_{l , t}} ( {thres}_{i , l} - {sm}_{i , l , t} ) \cdot {s}_{i}}{D \cdot {\sum }_{i = 1}^{{cluster}_{l , t}} {s}_{i}} ) )$$

where i represents a voxel; t represents a time; D represents a duration; l represents a soil layer; d represents the thickness of a soil layer; s represents an area of the grid cell; thres represents the drought threshold; sm represents the soil moisture value; and cluster represents a drought event.

These four-dimensional drought events are classified into three drought types, namely surface drought of a heavy-top inverted-iceberg-shaped structure, deep drought of a heavy-bottom iceberg-shaped structure, and a cylindrical drought structure when both surface drought and deep drought do not happen, where the surface drought is defined as that a duration of a surface soil moisture deficit area being larger than a deep soil moisture deficit area is longer than 60% of a total duration; the deep drought is defined as that a duration of the deep soil moisture deficit area being larger than the surface soil moisture deficit area is longer than 60% of the total duration. In the classification process, uppermost two layers (0-7 cm and 7-28 cm) are selected as surface soils, and a third layer (28-100 cm) is selected as deep soil.

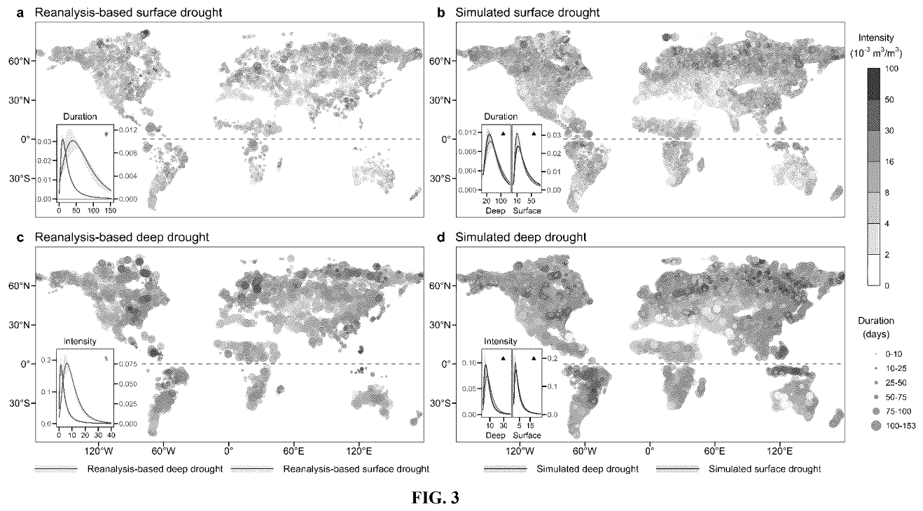

During 1981-2020, as shown in FIG. 3, 6065 consecutive four-dimensional drought events were identified and classified into surface drought and deep drought based on whether they exhibited the heavy-top inverted-iceberg-shaped structure or the heavy-bottom iceberg-shaped structure. Surface drought is characterized by a larger surface soil moisture deficit area and a smaller deep drought area, resembling an inverted-iceberg shape. In contrast, deep drought is characterized by a larger deep soil moisture deficit area than that of surface drought, resembling an iceberg shape. Globally, there were 3557 surface drought events, accounting for 58.7% of the total drought events, and 1655 deep drought events occurred, accounting for 27.3% of the total drought events. Deep drought is more persistent and intense than surface drought, particularly in the Amazon Basin, Africa, North America, East Asia, and high-latitude regions of the Northern Hemisphere.

### (4) Detection and Attribution of Four-Dimensional Soil Moisture Drought Indicators

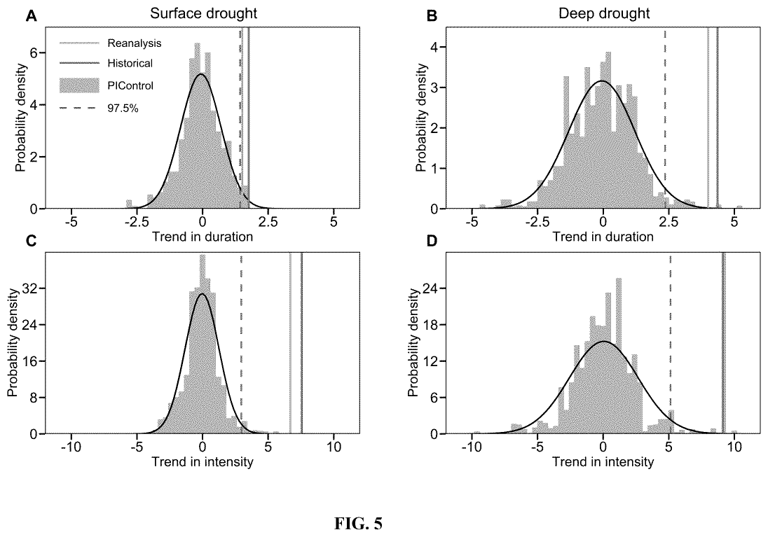

In this implementation, the annual average of durations and intensities of global surface drought and deep drought are calculated based on reanalysis (an average of three reanalysis datasets) and CMIP6 historical experiment simulation (the multi-model average), thereby obtaining an indicator sequence of the durations and intensities of global surface drought and deep drought. During 1981-2020, both the duration and intensity of surface drought showed a significant upward trend (p<0.01), and the growth rates of the duration and intensity of deep drought were much higher than those of surface drought. The trends in the durations and intensities of surface drought and deep drought based on reanalysis data fall outside the range of 2.5%-97.5% of the trends from 2000 drought events of 40 years based on pre-industrial control simulation (FIG. 5). This indicates that the increase in the characteristic indicators of surface drought and deep drought cannot be fully explained by internal climatic variability. When anthropogenic impacts are considered, the CMIP6 historical experiment simulation can well capture the upward trends of the characteristic indicators of these two types of drought, as well as the faster growth rate of deep drought.

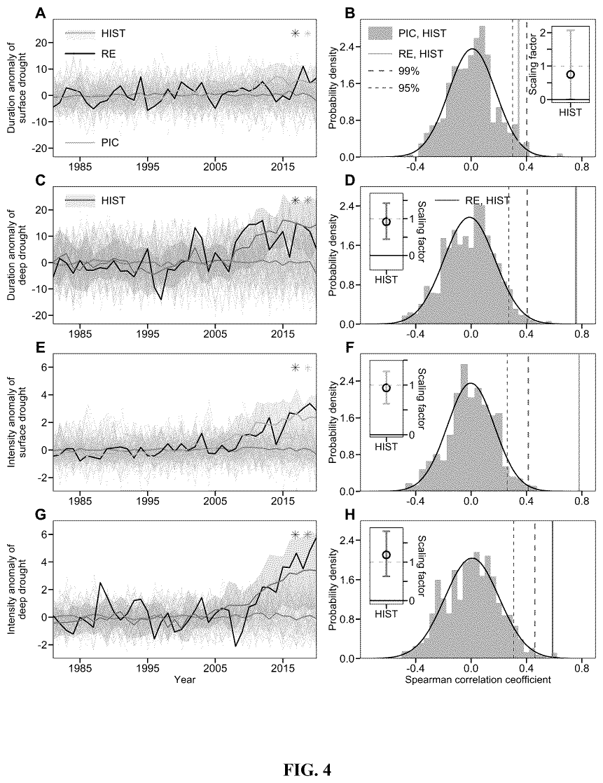

Two detection and attribution methods (the correlation coefficient method and the optimal fingerprint method) are adopted to quantitatively assess the impact of anthropogenic warming on the changes in the two types of global four-dimensional drought events. Based on the correlation coefficient method, the Spearman correlation coefficient between changes in duration and intensity characteristic sequences of yearly global surface drought and deep drought based on reanalysis (the average of three reanalysis data sets) and CMIP6 historical experiment simulation (the multi-model average). Moreover, the Spearman correlation coefficients between the multi-model averages of CMIP6 historical experiment simulations for two types of drought indicator sequences and 2000 overlapping 40-year data chunks simulated through pre-industrial experiment simulation are calculated (FIG. 4). In this implementation, under the historical experiment simulation conditions, the time sequences of the two characteristic indicators of surface drought show a significant correlation (p<0.05) with the time sequences based on reanalysis datasets. The correlation of deep drought between the historical experiment simulation and reanalysis is greater than the 99th percentiles of all correlations between the historical experiment simulation and the pre-industrial experiment simulation (FIG. 4), confirming that anthropogenic emissions have led to the intensification of drought throughout the vertical soil profile.

The results based on the correlation coefficient method are verified by using the regularized optimal fingerprint method, and the difference between reanalysis and the historical experiment simulation is quantified. Specific calculation is as follows:

$$Y = ( X - α ) β + ε$$

where Y represents a global average characteristic change of surface drought and deep drought based on reanalysis; X represents a global average characteristic change of surface drought and deep drought based on model simulation; α represents sampling uncertainty; β represents a scaling factor; ε represents an internal climatic variability estimated through the pre-industrial experiment simulation. To reduce the noise caused by interannual variations, the characteristic sequences of surface drought and deep drought are averaged at intervals of 3 consecutive years; if both the scaling factor and its 90% confidence interval are greater than zero, the external forcing signal is considered detectable at the 5% significance level. That is, the anthropogenic warming signal can be detected.

As shown in the right column of FIG. 4, the impact of historical external forcing can be detected in the duration and intensity increases of the two types of drought events. This verifies the results based on the correlation coefficient, i.e., at a confidence level of at least 90% (5%-95%), and anthropogenic emissions have caused the temporal changes in the characteristics of the two types of drought. The scaling factor values of the characteristics of the two types of drought are close to 1, indicating that the models can accurately reproduce the changes in most of drought characteristics based on reanalysis.

This implementation shows that during 1981-2020, anthropogenic climate change has most likely (90% probability) led to increases in the durations and intensities of global surface drought and deep drought. The strong external signal (mainly anthropogenic emissions) in the temporal change of deep drought further indicates that the impacts of anthropogenic factors on global soil moisture drought have penetrated into deeper soil layers below the surface.

### (5) Estimation of Future Evolution of Four-Dimensional Soil Moisture Drought

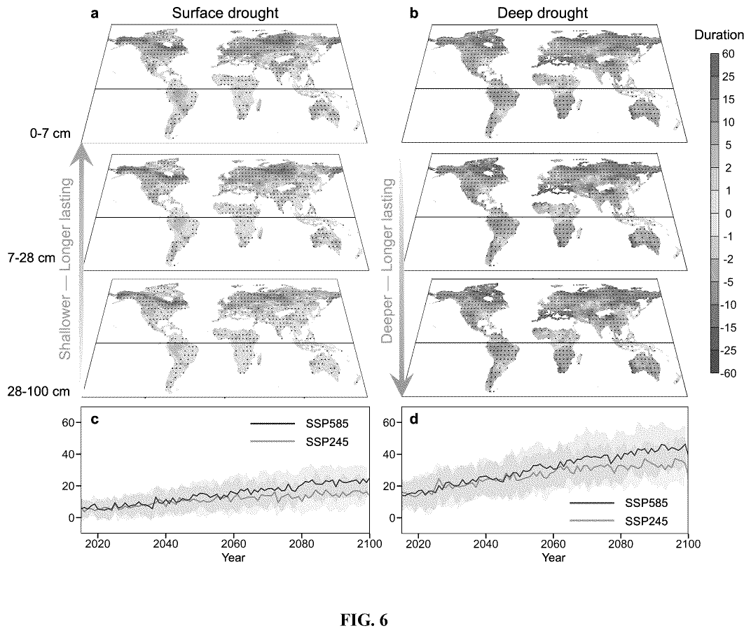

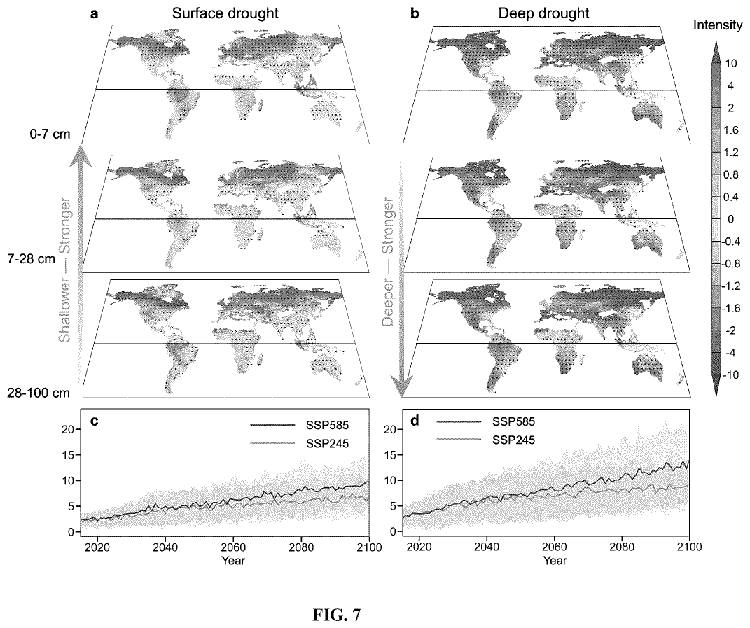

In this implementation, the spatial-temporal evolution of global surface drought and deep drought characteristics in the future period is estimated by using the CMIP6 historical experiment simulation and the representative concentration pathway and the shared socio-economic pathway (SSP) simulations in the future scenarios including SSP245 and SSP585 scenarios. As shown in FIG. 6, in the SSP245 scenario, compared with the historical period (1981-2020), the estimated increase in the duration of surface drought in the future period (2061-2100) will mainly occur in the high-latitude regions in the Northern Hemisphere (30° N or above), and the intensity of surface drought in these regions is expected to increase by 335.9% (FIG. 7). In contrast, more persistent (+154.9%) and more intense (+477.3%) deep drought is expected to occur across the global land, particularly in the Amazon Basin, southern Africa, Australia, southeastern North America, the Middle East, and the high-latitude regions in the Northern Hemisphere (FIG. 6 and FIG. 7). In the vertical soil profile, the shallower the soil depth, the greater the increase amplitude of the two characteristics of surface drought; whereas the deeper the soil depth, the greater the increase amplitude of deep drought. This indicates that under the context of climate warming, the duration and intensity of deep (surface) drought are expected to intensify starting from the bottom (top). In the SSP585 scenario, the above-mentioned increase amplitudes of surface drought and deep drought are even greater.

As shown in FIG. 6 and FIG. 7, the temporal change trends of the characteristics of the two types of drought events also show a significant upward trend. In the SSP245 scenario, the duration of surface drought is expected to increase by 1.3 days per decade, while its intensity is expected to increase by 5.4×10⁻⁴ m³·m⁻³ per decade. The increase amplitudes of the two characteristics of deep drought (duration: an increase of 2.4 days per decade; and intensity: an increase of 6.5×10⁻⁴ m³·m⁻³ per decade) are greater than those of surface drought. Under the influence of increased anthropogenic emissions, the increase amplitudes of surface drought and deep drought in the SSP585 scenario are further amplified by 1.6 to 2.0 times. This indicates that the duration of global deep drought will be longer, its intensity will be greater, and the risk of drought intensification in deeper soil will also be higher.

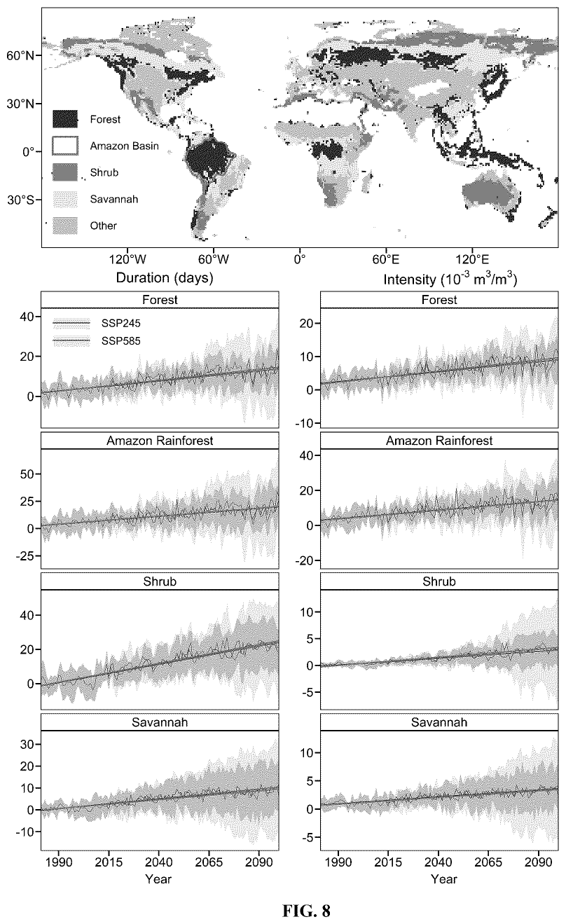

Changes of surface drought and deep drought characteristics of different vegetation types are estimated in combination with a land cover dataset; deep-rooted vegetation cover categories (i.e., forest, shrub, and savannah) are selected, and a weighted area average of deep drought and surface drought characteristic (duration and intensity) differences of different vegetation regions is calculated. As shown in FIG. 8, for ecosystems known to contain deep-rooted species (such as forests, shrubs, and savannahs), the differences in duration and intensity between deep drought and surface drought during 1981-2100 show a significant upward trend, and an increase in such differences is also observed in the Amazon Basin.

### (6) Physical Driving Factors of Surface Drought and Deep Drought

In this implementation, surface drought and deep drought events are attributed to different driving factors, such as extreme high temperature (TEM), precipitation deficit (PRE), and vegetation greening (high leaf area index: LAI), an average of three variables in 30 days before a drought event is calculated, and a severity index (SI) of the drought event is calculated, with specific calculation as follows:

$${SI}_{v} = ( {v}_{i , j} - {μ}_{j} ) / {σ}_{j}$$

where i represents a year; j represents a day; v represents a driving factor; and μ and σ represent a climatological normal and a standard deviation of a climatological (historical period: 1981-2010, and future period: 2061-2100) variable, respectively.

The extreme high temperature is defined as SI_{TEM}>0.8, the precipitation deficit is defined as SI_{PRE}<−0.8, and the vegetation greening is defined as SI_{LAI}>0.8; if a drought event and an extreme event occurring in 30 days before the drought event appear at a same grid cell, the drought event at the grid cell is attributed to the driving factor of this type; and normalized contributions of different driving factors to durations and intensities of the two types of drought events are calculated.

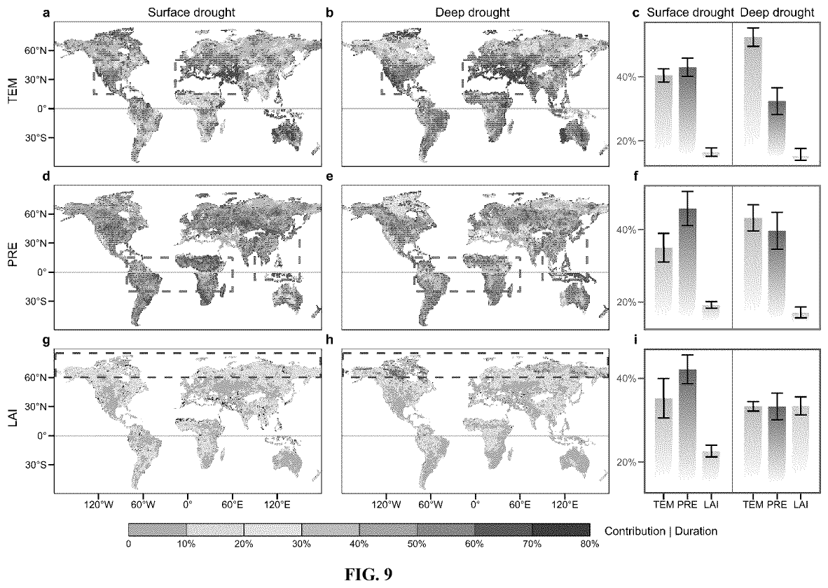

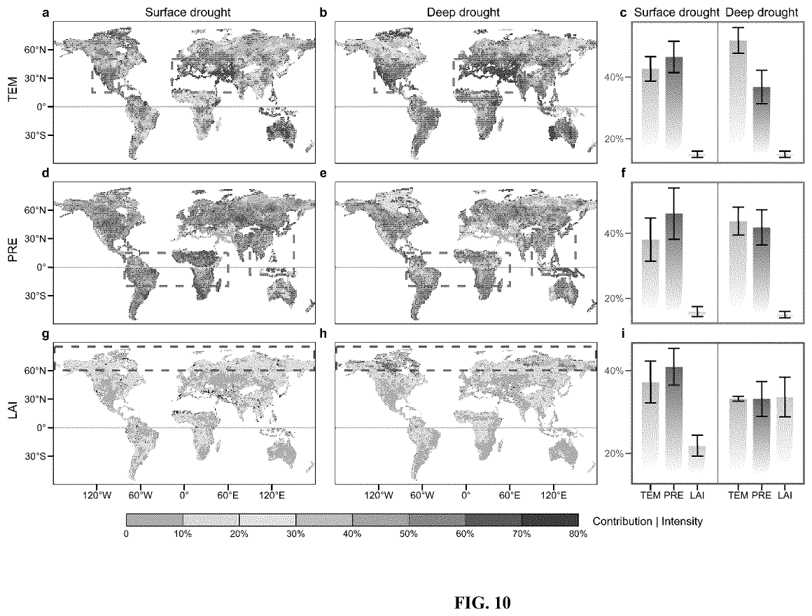

As shown in FIG. 9 and FIG. 10, based on three reanalysis datasets of 1981-2020, the early-stage precipitation deficits account for 45% and 44% of the total duration and intensity of global surface drought, respectively, and dominate the evolution of 69% and 61% of global surface drought, respectively. This indicates that global surface drought is highly sensitive to precipitation changes. However, the dominant driving factor of deep drought exhibits significant spatial heterogeneity. In droughty regions, the extreme high temperature at the early stage is the dominant driving factor of deep drought, particularly in eastern North America, Europe, and the Middle East, accounting for 52% of the total duration and intensity. These regions are hotspots for soil moisture-air temperature coupling.

In humid regions, such as the tropics and eastern Asia, the total duration and intensity of deep drought are mainly attributed to the concurrent occurrence of early-stage precipitation deficits (40% and 42%) and extreme high temperatures (43% and 44%). In global vegetation greening hotspots, such as the high-latitude regions of the Northern Hemisphere, the role of early-stage vegetation greening cannot be ignored, with each of the three aforementioned driving factors contributing 33% (see i in FIG. 9 and i in FIG. 10).

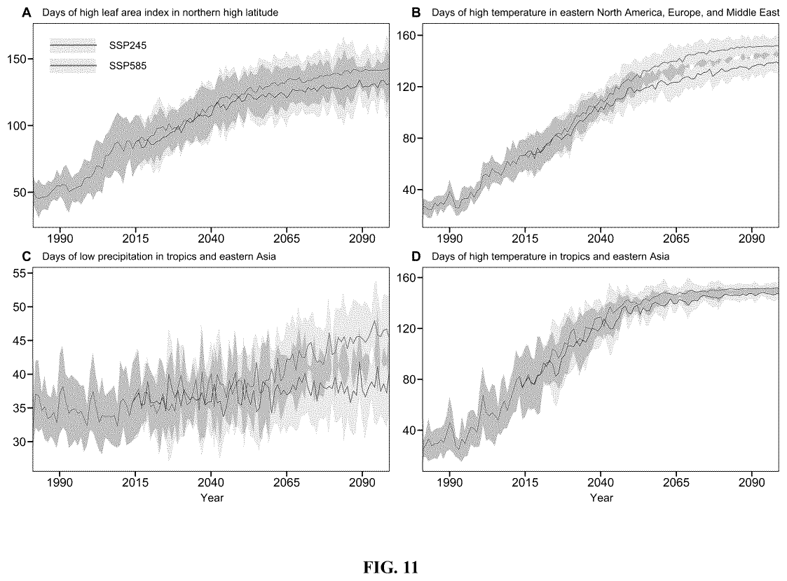

As shown in FIG. 11, during 2061-2100, under the scenario of sustained anthropogenic emissions, the dominant driving factors of drought in different climatic regions will change slightly. The number of extreme high-temperature days in eastern North America, Europe, and the Middle East is expected to increase; the concurrent occurrence of precipitation deficit and extreme high temperature in the tropics and eastern Asia will become more frequent; and vegetation greening in the high-latitude regions of the Northern Hemisphere will become more persistent. Anthropogenic climate change will intensify these driving processes, further exacerbating deep drought.

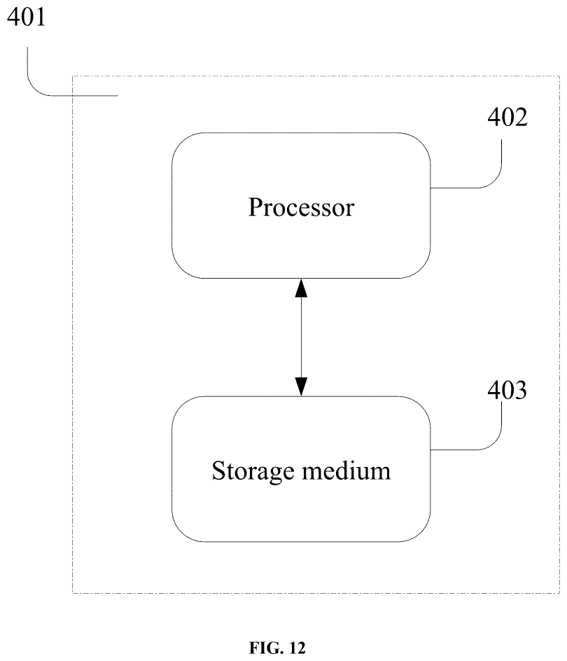

FIG. 12 is a schematic diagram of working of a hardware device according to an embodiment of the present disclosure. Referring to FIG. 12, the hardware device specifically includes a device 401 for tracking a four-dimensional soil moisture drought event and identifying a warming signal, a processor 402, and a storage device 403.

The device 401 for tracking a four-dimensional soil moisture drought event and identifying a warming signal is configured to implement the method for tracking a four-dimensional soil moisture drought event and identifying a warming signal.

The processor 402 is configured to load and execute instructions and data in the storage medium 403 to implement the method for tracking a four-dimensional soil moisture drought event and identifying a warming signal.

The storage medium 403 is configured to store instructions and data and to implement the method for tracking a four-dimensional soil moisture drought event and identifying a warming signal.

The present disclosure has the following beneficial effects.

(1) The present disclosure provides a method for identifying four-dimensional soil moisture drought, which helps to deepen the understanding of this new type of soil moisture drought for the academic community. In the present disclosure, the four-dimensional soil moisture drought is classified into two different types; the spatial-temporal characteristic indicators of the four-dimensional soil moisture drought are quantified; and the changes and impacts of the four-dimensional soil moisture drought in the four-dimensional spatial-temporal domain (including longitude, latitude, depth, and time) are clarified. In the present disclosure, the differences in spatial-temporal evolution of two types of four-dimensional soil moisture drought under different land cover types in future periods are quantified, providing a scientific basis for in-depth understanding of the dynamic evolution process of drought events.

(2) In the present disclosure, quantitative anthropogenic warming signal detection is performed on the spatial-temporal evolution process of the four-dimensional soil moisture drought characteristics, the impacts of anthropogenic warming on the changes of the two types of four-dimensional drought events are identified, and the changes of four-dimensional drought events in the future are estimated. It provides theoretical support for decision-makers to make decisions for adapting to and mitigating the impact of climate change on soil moisture drought.

(3) In the present disclosure, the contributions of the driving factors of the four-dimensional soil moisture drought events are analyzed; the contributions of different driving factors to four-dimensional soil moisture drought in historical and future periods are clarified; the dominant driving factors of the two types of four-dimensional soil moisture drought are quantified; theoretical support is provided for the attribution of four-dimensional soil moisture drought.

The foregoing are merely descriptions of the preferred embodiments of the present disclosure, and are not intended to limit the present disclosure. Any modifications, equivalent replacements and improvements made within the spirit and principle of the present disclosure should be included in the protection scope of the present disclosure.

What is claimed is:

1. A method for tracking a four-dimensional soil moisture drought event and identifying a warming signal, comprising following steps:
step S1, data acquisition: acquiring global climate data;
step S2, identification of four-dimensional soil moisture drought events: calculating, in combination with the data obtained in the step S1, a 10th percentile of a soil moisture value during a growing season of a climatological period for each voxel as a drought threshold, identifying voxels for which soil moisture values are lower than the drought threshold as drought voxels, and identifying continuous four-dimensional soil moisture drought events by a Lagrangian four-dimensional tracking method;
step S3, characteristic quantification of four-dimensional soil moisture drought events: calculating characteristics of soil moisture drought events in combination with the four-dimensional soil moisture drought events obtained in the step S2, and classifying the soil moisture drought events into surface drought and deep drought;
step S4: anthropogenic warming signal detection based on temporal evolution of four-dimensional soil moisture drought characteristics in a historical period: performing anthropogenic warming signal detection on drought characteristic indicators calculated based on reanalysis data and historical climatic experiments of climate models by using a correlation coefficient method and an optimal fingerprint method in combination with duration and intensity characteristics of two types of drought events obtained in the step S3;
step S5: estimation of future evolution of four-dimensional soil moisture drought: obtaining spatial-temporal evolution characteristics of drought events in a future period through the historical climatic experiments of the climate model and a future socio-economic development pathway experiment in combination with the duration and intensity characteristics of the two types of drought events obtained in the step S3; and obtaining spatial-temporal evolution characteristics of drought events under different vegetation types in the future period in combination with global land cover data obtained in the step S1; and
step S6: attribution of four-dimensional soil moisture drought events to driving factors: attributing the two types of drought events to three early-stage driving factors according to data of near-surface air temperature, precipitation, and leaf area index obtained in the step S1 in combination with the duration and intensity characteristics of the two types of drought events obtained in the step S3, and quantifying contributions of different driving factors to the drought events.

2. The method according to claim 1, wherein the global climate data in the step S1 comprises: soil moisture content, near-surface air temperature, and precipitation data of three reanalysis datasets from a fifth generation land surface reanalysis dataset of European Centre for Medium-Range Weather Forecasts (ERA5-Land), global atmosphere and land surface reanalysis product data of China Meteorological Administration (CRA-Land), Global Land Data Assimilation System Noah model (GLDAS-Noah), leaf area index data of Global Long-Term Leaf Area Index (GLOBMAP) data, acquired precipitation data of Global Soil Moisture Project Phase 3 (GSWP-3), acquired soil moisture content, near-surface air temperature, precipitation, and leaf area index data of Coupled Model Intercomparison Project Phase 6 (CMIP6), and acquired global land cover data of Moderate Resolution Imaging Spectroradiometer (MODIS) Land Cover Climate Modeling Grid (MCD12C1).

3. The method according to claim 1, wherein the step S2 specifically comprises:
S21, preprocessing of soil moisture data: removing grid cells of regions of the south and north poles and desert regions with an annual precipitation of less than 100 mm according to GSWP-3 precipitation data; using a soil moisture value in a growing season; converting units of the soil moisture value from mass units to volume units; interpolating multi-layer soil moisture values of different data to a common vertical profile; converting datasets on different time scales to datasets on a day scale; interpolating the datasets with a resolution 1°×1° by second-order conservative remapping, and ensuring a consistent temporal-spatial resolution; and eliminating abnormal sequence fluctuations by 11-day smoothing averaging; and
S22, simulation of spatial-temporal evolution of soil moisture drought in four dimensions by using the Lagrangian tracking method:
for each grid cell of a single layer, when the soil moisture value is lower than the 10th percentile of the soil moisture value during the growing season of the climatological period, defining the grid cell as the drought grid cell; combining adjacent drought grid cells in the single layer into consecutive drought patches in a two-dimensional space; for each time step, joining all adjacent two-dimensional drought patches in 26 adjacent areas into an independent three-dimensional consecutive drought patch; and if an overlapping ratio of any spatially consecutive three-dimensional drought patches is greater than a preset percentage in consecutive time steps, combining the spatially consecutive three-dimensional drought patches into a single four-dimensional drought event, wherein the four-dimensional drought event lasts at least for a preset number of days, and a projection area is greater than a preset value.

4. The method according to claim 1, wherein the step S3 specifically comprises:
S31, spatial and temporal characteristics of each of the four-dimensional soil moisture drought events being a duration and an intensity, wherein the duration refers to a duration of the four-dimensional soil moisture drought event; and the intensity is a weighted average of an area and a voxel with soil moisture deficit among all voxels in the four-dimensional soil moisture drought event, and is expressed as follows:
$$Intensity = \frac{{d}_{l}}{{\sum }_{l = 1}^{k} {d}_{l}} ( {\sum }_{l = 1}^{k} ( \frac{{\sum }_{t = {t}_{0}}^{D} {\sum }_{i = 1}^{{cluster}_{l , t}} ( {thres}_{i , l} - {sm}_{i , l , t} ) \cdot {s}_{i}}{D \cdot {\sum }_{i = 1}^{{cluster}_{l , t}} {s}_{i}} ) )$$
wherein i represents a voxel; t represents a time; D represents a duration; l represents a soil layer; d represents the thickness of a soil layer; s represents an area of the grid cell; thres represents the drought threshold; sm represents the soil moisture value; and cluster represents a drought event;
S32, classifying four-dimensional drought events into three drought types, namely surface drought of a heavy-top inverted-iceberg-shaped structure, deep drought of a heavy-bottom iceberg-shaped structure, and a cylindrical drought structure when both surface drought and deep drought do not happen, wherein the surface drought is defined as that a duration of a surface soil moisture deficit area being larger than a deep soil moisture deficit area is longer than a preset percentage of a total duration; the deep drought is defined as that a duration of the deep soil moisture deficit area being larger than the surface soil moisture deficit area is longer than the preset percentage of the total duration; and
S33, in a classification process, selecting uppermost two layers as surface soil and a third layer as deep soil.

5. The method according to claim 1, wherein the step S4 specifically comprises:
S41, performing anthropogenic climate change detection and attribution on the four-dimensional soil moisture drought events based on the correlation coefficient method, and calculating a Spearman correlation coefficient between changes of global surface drought and deep drought characteristic sequences based on the reanalysis data and CMIP6 historical experiment simulation; dividing a CMIP6 pre-industrial experiment simulation into a plurality of data chunks for same years as a research period, and calculating the Spearman correlation coefficient between the plurality of data chunks and a multi-model average of historical experiment simulation; and analyzing whether a correlation coefficient between the reanalysis data and a drought event characteristic sequence of the historical experiment simulation exceeds a 95th or 99th percentile of a correlation coefficient set between the historical experiment simulation and a plurality of data chunks of the pre-industrial experiment simulation; and
S42, performing anthropogenic climate change detection and attribution on the four-dimensional soil moisture drought events based on the optimal fingerprint method, with specific calculation as follows:
$$Y = ( X - α ) β + ε$$
wherein Y represents a global average characteristic change of surface drought and deep drought based on reanalysis; X represents a global average characteristic change of surface drought and deep drought based on model simulation; α represents sampling uncertainty; β represents a scaling factor; ε represents an internal climatic variability estimated through the pre-industrial experiment simulation.

6. The method according to claim 1, wherein the step S5 specifically comprises:
S51, calculating the spatial-temporal evolution of global surface drought and deep drought characteristics in the future period by using a CMIP6 historical simulation experiment and a representative concentration pathway and a shared socio-economic pathway (SSP) simulations in future scenarios comprising SSP245 and SSP585 scenarios; and
S52, estimating changes of surface drought and deep drought characteristics of different vegetation types in combination with a land cover dataset, selecting a deep-rooted vegetation cover category, and calculating a weighted area average of deep drought and surface drought characteristic differences of different vegetation regions.

7. The method according to claim 1, wherein the step S6 specifically comprises:
S61, attributing surface drought and deep drought events to three different driving factors, namely extreme high temperature (TEM), precipitation deficit (PRE), and high leaf area index (LAI) from vegetation greening, calculating an average of three variables in 30 days before a drought event, and calculating a severity index (SI) of the drought event, with specific calculation as follows:
$${SI}_{v} = ( {v}_{i , j} - {μ}_{j} ) / {σ}_{j}$$
wherein i represents a year; j represents a day; v represents a driving factor; and μ and σ represent a climatological normal and a standard deviation of a climatological variable, respectively; and
S62, defining the extreme high temperature as SI_{TEM}>a preset value, the precipitation deficit as SI_{PRE}<a preset value, and the vegetation greening as SI_{LAI}>a preset value; if a drought event and an extreme event occurring in 30 days before the drought event appear at a same grid cell, attributing the drought event at the grid cell to the driving factor of the extreme high temperature type; and calculating contributions of different driving factors to durations and intensities of the two types of drought events.

8. A non-transitory storage medium, storing instructions and data for implementing the method for tracking a four-dimensional soil moisture drought event and identifying a warming signal according to claim 1.

9. A device for tracking a four-dimensional soil moisture drought event and identifying a warming signal, comprising a processor and a storage medium, wherein the processor is configured to load and execute instructions and data in the storage medium to implement the method for tracking a four-dimensional soil moisture drought event and identifying a warming signal according to claim 1.
